(19) United States
(12) Patent Application Publication
(10) Pub. No.: US 2006/0234993 A1
(43) Pub. Date: Oct. 19, 2006

(54) Use of androstane derivatives for enhancing physical performance

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave., Detroit, MI, 48226, US

(21) Appl. No.: 11/403,641

(22) Filed: Apr. 13, 2006

Related U.S. Application Data
(60) Provisional application No. 60/670,637, filed on Apr. 13, 2005.

(51) Int. Cl. A61K 31/57

(52) U.S. Cl. 514/177; 514/178

(57) ABSTRACT

A method of decreasing body weight, reducing adipose tissue and reducing appetite in humans comprises administering a 5-Beta androstane derivative or a physiologically acceptable salt, ester or ether thereof of the general formula: 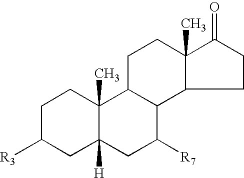 wherein R3 is one of α-OH , β-OH, a mixture of α-OH and , β-OH, and O; and R7 is one of α-OH, β-OH and O.

### RELATED APPLICATION

This application claims priority benefit of U.S. provisional patent application No. 60/670,637 filed on Apr. 13, 2005.

### FIELD OF THE INVENTION

This invention relates to use of androstane derivatives for decreasing body weight, reducing adipose tissue and reducing appetite.

### BACKGROUND OF THE INVENTION

Obesity and increased body weight are growing concerns in the United States as these conditions lead to a variety of health problems such as hypertension, heart disease, atherosclerosis, diabetes and cancer. Weight loss, specifically fat loss, is critical in maintaining a healthy body weight. It is desirable to maintain relatively high lean body mass (i.e., muscle mass) as a percentage of total body mass for overall good health. Benefits of avoiding obesity and maintaining lean body mass are well known, and include well-regulated metabolism, proper maintenance of body adiposity, enhanced glucose disposal and regulation of glucose levels. In addition, the maintenance of lean body mass is also associated with increased self-esteem.

There are several known strategies for maintaining or decreasing body weight, reducing adipose tissue and reducing appetite through the use of pharmaceuticals and dietary supplements. For example, ephedrine and amphetamines have been used extensively for weight loss. The use ephedrine and amphetamines, while shown to be effective in the prior art, is associated with increased risk of side effects including increased heart rate and blood pressure due to over stimulation of beta-adrenergic receptors.

Etiocholanolone, or 3-alpha (beta)-hydroxy-5β-androstan-17-one has been shown to reduce body weight in obese mice and in normal mice. In addition, 7-oxo and 7-hydroxy derivatives of dehydroepiandrosterone (DHEA) have also been shown to have similar effects. Such known androstane derivatives, however, have been shown to be rapidly metabolized and excreted. It is believed that etiocholanolone is rapidly conjugated (made hydrophilic) at the 3-hydroxyl group resulting in a polar, excretable molecule. DHEA and its derivatives are not desirable in that they have been shown to have negative estrogenic and androgenic side effects, including gynecomastia, water retention (edema), and fat retention, prostate enlargement, acne, and hair loss. It would be desirable to provide androstane derivatives which last longer in the body with reduced negative effects.

### SUMMARY OF THE INVENTION

In accordance with a first aspect, a method of decreasing body weight, reducing adipose tissue and reducing appetite in humans comprises administering a 5-beta androstane derivative. The 5-beta androstane derivative can comprise, for example, 3-alpha (beta)-hydroxy-5βandrostan-7,17-dione.

From the foregoing disclosure and the following more detailed description of various preferred embodiments it will be apparent to those skilled in the art that the present invention provides a significant advance in the methods for decreasing body weight, especially reducing adipose tissue. Additional features and advantages of various preferred embodiments will be better understood in view of the detailed description provided below.

### DETAILED DESCRIPTION OF CERTAIN PREFERRED EMBODIMENTS

It will be apparent to those skilled in the art, that is, to those who have knowledge or experience in this area of technology that many variations are possible for the method of reducing body weight disclosed here. The following detailed discussion of various alternative and preferred features and embodiments will illustrate the general principles of the invention with reference to improved method of reducing body weight and adipose tissue through the use of orally available dietary supplements. Other embodiments suitable for other applications will be apparent to those skilled in the art given the benefit of this disclosure.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance. Here, for example, the parent compound is an androstane derivative, also sometime referred to as etiocholanolone, 3-alpha(beta)-etiocholan-17-one or 3-alpha (beta)-hydroxy-5β-androstan-17-one. A derivative may be made by modification of the parent compound or by synthesis from other starting materials that are not similar to the parent.

Preferably the androstane derivative which is relatively long lasting in vivo is of the general formula:

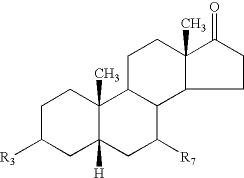

wherein R3 is one of α-OH, , β-OH, a mixture of α-OH and , β-OH, and O band R7 is one of α-OH, , β-OH and O, in accordance with conventional steroid carbon numbering, an atom or functional group attached to a ring depicted here is termed α (alpha) if it lies below the plane of the paper or β(beta) if it lies above the plane of the paper. When R3 and/or R7 are OH, it is understood that the fourth bond to the carbon is hydrogen. Androstane derivatives made according to the above formula can comprise, for example: 3-alpha(beta)-hydroxy-5β-androstan-7,17-dione, 5-beta-7-beta-hydroxy androstan -3,17-dione, 5-beta,7-alpha-hydroxy androstan-3,17-dione, 5-beta-androstan-3,7,17-trione, 3,7-alpha-dihydroxy, 5-beta-androstan-17-one, 3,7-beta-dihydroxy-5-beta-androstan-17-one, 3-alpha-hydroxy-5-beta-7-beta-dihydroxy-androstan-17-one, 3-beta-hydroxy-5-beta-7-alpha-dihydroxy-androstan-17-one, as well as physiologically acceptable salts, esters or ethers thereof. For those compounds having R7=O, the presence of a 7-keto group allows 3-alpha (beta)hydroxy-5-beta-androstan-7,17-dione to act as a pro-hormone that is metabolized to 7-hydroxy androstane derivatives in the body. Other androstane derivatives suitable for reducing fat tissue will be readily apparent to those skilled in the art, given the benefit of this disclosure.

Advantageously, addition of the O or OH group at the R7 position and O at the R17 position increases the amount of useful 7-oxo derivatives formed in vivo. All of the naturally occurring androstane derivative compounds disclosed herein would preferably be administered orally mixed with solid or liquid carriers in appropriate unit doses for the purpose of decreasing body weight, reducing adipose tissue and reducing appetite.

The preferred amount of the active ingredient that is to be administered, such as orally would depends on various factors such as the age and weight of the user. An effective oral daily dosage of the described derivatives can comprise 10 to 500 mg daily, and most preferably about 50 to 150 mg daily. A preferred embodiment might be to administer the oral dose as a soft gelatin capsule or oral liquid suspension, either in two to three divided doses per day (i.e., 25 to 75 mg twice per day, or 12.5 mg to 37.5 mg four times per day). Androstane derivatives as disclosed herein may also be administered transdermally using acceptable liquid vehicles, sublingually, transrectally (by suppository) intranasally, intravenously, subcutaneously, or by intramuscular injection. Androstane derivatives as disclosed herein may also be mixed with dietary supplements such as green tea, if desired.

### EXAMPLE 1

Capsules. 1 kg of the androstane derivative, 3-alpha (beta)-hydroxy-5-beta-androstan-7,17-dione are mixed with microcrystalline cellulose, and placed into 100,000 hard-gelatin capsules. Each capsule contains 100 mg of 3-alpha (beta)-androstan-7,17-dione.

### EXAMPLE 2

Capsules. 1 kg of 3-alpha(beta)hydroxy-5-beta-androstan-7,17-dione is mixed with 5 kg of dry green tea, and placed into 10,000 hard-gelatin capsules. Each capsule contains 100 mg of 3-alpha (beta)-androstan-7,17-dione and 500 mg green tea.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of decreasing body weight in humans comprises administering a 5-beta androstane derivative or a physiologically acceptable salt, ester or ether thereof of the general formula: 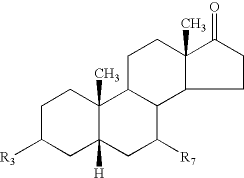
wherein
R3 is one of α-OH , β-OH, a mixture of α-OH and β-OH, and O; and
R7 is one of α-OH, β-OH and O.

2. The method of claim 1 wherein the androstane derivative is administered in one of the following ways: orally, transdermally, intranasally, by injection, sublingually, and transrectally.

3. The method of claim 1 wherein the androstane derivative is administered as a daily dosage between about 10 mg and 500 mg.

4. The method of claim 1 wherein the androstane derivative is administered as a daily dosage between about 50 mg and 150 mg.

5. The method of claim 1 wherein the androstane derivative is administered as a daily dosage between about 25 mg to 75 mg twice per day.

6. The method of claim 1 wherein the androstane derivative is administered as a daily dosage between about 12.5 mg to 37.5 mg four times per day.

7. The method of claim 1 wherein the androstane derivative is administered in the form of a gel capsule.

8. The method of claim 1 wherein R3 is a mixture of α-OH and β-OH and R7 is O.

9. The method of claim 1 wherein R3 is O.

10. The method of claim 9 wherein R7 is α-OH.

11. The method of claim 9 wherein R7 is β-OH.

12. The method of claim 9 wherein R7 is O.

13. The method of claim 1 wherein R3 is α-OH.

14. The method of claim 13 wherein R7 is α-OH.

15. The method of claim 13 wherein R7 is β-OH.

16. The method of claim 1 wherein R3 is β-OH.

17. The method of claim 16 wherein R7 is α-OH.

18. The method of claim 16 wherein R7 is β-OH.

19. The method of claim 1 further comprising administering the 5-beta androstane derivative with green tea.
